(19) United States
(12) Patent Application Publication
(10) Pub. No.: US 2006/0025683 A1
(43) Pub. Date: Feb. 2, 2006

(54) Hand-held imaging probe for treatment of states of low blood perfusion

(75) Inventor: Andrew Kenneth Hoffmann, Surrey (CA)

Correspondence Address: BORDEN LADNER GERVAIS LLP, WORLD EXCHANGE PLAZA, 100 QUEEN STREET SUITE 1100, OTTAWA, ON, K1P 1J9, CA

(73) Assignee: AHOF BIOPHYSICAL SYSTEMS INC.

(21) Appl. No.: 11/036,386

(22) Filed: Jan. 18, 2005

Related U.S. Application Data
(63) Continuation-in-part of application No. 10/902,122, filed on Jul. 30, 2004.

(51) Int. Cl. A61B 8/12

(52) U.S. Cl. 600/439

(57) ABSTRACT

A non-invasive hand-held treatment imaging probe for treatment of acute blood flow disturbances and states of low blood perfusion. The treatment imaging probe is operable to emit high intensity therapeutic mechanical acoustic waves while under ultrasonic imaging guidance. The probe has a substantially rigid application surface, sized to enable seating within a rib space of a patient (i.e. for cardiac applications), comprising the combination of an engagement face of an ultrasonic imaging transducer and an application end of a high powered therapeutic actuator operable in about the 1-500 kHz (and preferably 1-150 kHz) range. Treatment imaging probe can be used as an adjunct to thrombolytic therapy in the treatment of acute thrombotic vascular obstructions, such as in acute myocardial infarction, or alternatively to enhance localized delivery of angiogenic agents or other useful medications to targeted vascular regions.

### CROSS-REFERENCE TO RELATED APPLICATIONS

This application is a continuation-in-part of co-pending U.S. patent application Ser. No. 10/902,122, filed Jul. 30, 2004, the contents of which are expressly incorporated herein by reference.

### FIELD OF THE INVENTION

This invention relates to non-invasive hand-held imaging instruments adapted to impart therapeutic acoustic energy to a human body, for emergency treatment of acute blood flow disturbances.

### BACKGROUND OF THE INVENTION

Acute thrombosis, ischemia and coronary artery disease are all common concerns. Acute myocardial infarction (AMI) subsequent to coronary thromboses, in particular, is one of the leading causes of death in North America and Europe. Current first-line treatment of thromboses in the acute phase when the patient reaches professional care is typically by intravenous administration of thrombolytics, or a combination of drugs such as heparin, aspirin and/or GP 2b 3a platelet inhibitors to dissolve the blood clot. Intravenous and oral nitrates may also be introduced in order to dilate the culprit coronary vessel, which usually has a degree of spasm associated.

Thrombolytic drug treatment does not, however, have a high success rate, with adequate reperfusion occurring only between 50-63% of the time within ninety minutes of administration of the thrombolytic drug. Furthermore, success with drug based reperfusion treatment and in-hospital survival declines markedly when the patient becomes hemodynamically unstable or enters cardiogenic shock, which is the leading cause of in-hospital deaths from AMI in North America.

Treatment systems utilizing non-invasive mechanical vibration, or acoustic energy, have been employed as an adjunct to systemically delivered IV thrombolysis, including coronary thrombolysis, to improve these outcomes. Externally delivered acoustic energy provides mechanical agitation via cavitation and acoustic streaming to the blood within the culprit vasculature where a blood clot resides, thereby encouraging disruption of the clot and increased permeation of the drug into the clot to accelerate and ensure reperfusion. Furthermore, acoustic energy exhibits potent vasodilatory effects, which further aid in the restoration of flow. Acoustic energy in the high sonic to low ultrasonic frequency ranges, i.e. from about 1 kHz to about 150 kHz, is particularly desirable for treatment of blood flow disturbances, as acoustic energy in this frequency range is known for its superior penetration characteristics, clot disruptive capability, and enhancement of thrombolytic drug effectiveness.

Transcutaneously imparted therapeutic acoustic energy in this frequency range however, generally requires high intensity transmission levels and a high duty factor to ensure penetration and a therapeutic effect. These higher intensity requirements have tended to cause burning of overlying skin and soft tissue, and have thereby been problematic in practical use.

Prior methods for applying therapeutic non-invasive high sonic to low ultrasonic acoustic energy to a skin surface in the emergency treatment of acute blood flow disturbances have, therefore, typically used acoustic energy actuators placed upon specifically designed large bladders filled with acoustic coupling material to prevent skin overheating. High sonic to low ultrasonic frequency waves of relatively high intensity are thereby imparted through the bladder, such as via a divergent beam, to the target skin surface, enabling the non-specific delivery, or bathing, of the skin surface in the hopes that acoustic energy will penetrate to the culprit vasculature regions lying beneath the skin surface. It is well known however that overlying body tissue often blocks penetration of the acoustic waves as the waves are absorbed as heat or reflected and dissipated throughout the overlying body tissue, and thereby often never reach their intended vascular target, which is more deeply situated, especially in cardiac applications where the interference of dense overlying intercostal tissue and overlying lung does not transmit acoustic energy effectively.

It is therefore desirable to use an imaging modality to direct therapeutic acoustic energy in transcutaneous applications, to ensure adequate penetration and targeting of the therapeutic acoustic signal.

For clinical practicality, it has been shown that the placement of an ultrasonic imaging transducer, such as a phased array, in direct proximity to the application end of a therapeutic acoustic actuator, can be used effectively to establish an optimized acoustic penetration window, such as to ensure therapeutic penetration and targeting of the therapeutic signal.

Therapeutic acoustic energy is typically applied at different frequencies than those used in ultrasonic imaging. Specifically, it is desirable to apply therapeutic acoustic energy at lower frequencies, for example, from about 1 kHz to about 500 kHz, in order to achieve superior penetration, whereas higher frequencies, such as in the megahertz range, while much more prone to attenuation, are employed in diagnostic imaging to obtain better resolution.

Siegel et al., in U.S. Pat. No. 5,695,460, discloses a hand-held non-invasive actuator operational in the low ultrasonic ranges for chest wall placement in the emergency treatment of heart attacks, or coronary thrombosis. Siegel does not suggest the use of ultrasonic imaging, or any other form of medical imaging, to enable targeting of the disclosed actuator through a confirmed acoustic penetration window. Furthermore the actuator disclosed by Siegel is sub-optimal as it does not include a mechanism to prevent burning of the patient's skin, which is a common concern in high intensity, low ultrasonic frequency skin surface delivery.

Nock et al. in U.S. Patent Publication No; 2003/0204141, discloses an ultrasonic combined therapy/imaging system using the piezoelectric elements of a standard ultrasonic imaging transducer for imaging and therapy. The therapeutic pulses of ultrasound are provided at a higher intensity, or power, and/or duty cycle relative to the imaging cycles, hence causing selective heating of tissues within the target region imaged to cause a therapeutic result. Piezoelectric crystals and ultrasonic transmission systems designed for ultrasonic imaging operate in the higher frequency megahertz range, thus the therapeutic pulses are severely limited in penetration power and therapeutic effectiveness. Other examples of systems wherein ultrasonic imaging is combined with higher frequency therapeutic emitters, such as those that operate in the megahertz range, are also disclosed in U.S. Pat. Nos. 3,735,755 and 4,484,569.

Instruments comprising an ultrasonic imaging array coupled to a therapeutic actuator operable in the low ultrasonic range, such as where the two modalities are placed proximate one another, to enable directed therapy, have been disclosed in U.S. Pat. Nos. 5,391,140; 5,558,092; 5,873,828; 5,523,058; and PCT Publication No. WO 02/054018, for a variety of applications ranging from rectal treatments to adipose tissue disruption. None of the previously known instruments have a suitable application surface specifically sized and structured to enable efficient seating within a rib space of a patient, which can be of special importance to achieve optimal penetration in thoracic cavity, or cardiological applications, and none of the previously disclosed systems are equipped with the appropriate combination of high intensity emission capabilities, and means for limiting the heating of the contact surface, such as is required for external skin surface applications where deep penetration is generally required.

Acoustic disruptive techniques employing high intensity focussed ultrasonic shock waves have been employed successfully in lithotripsy, where a shock wave emitter is used with an ultrasonic positioning unit. Lithotripters require precise focussing of a target which is not possible in coronary thrombosis applications, as the coronary arteries cannot be imaged by non-invasive ultrasonic techniques, and the culprit blood clot thereby comprises a hidden, moving target. Furthermore, lithotripters are not designed to enable continuous wave, or high duty factor pulsed wave, acoustic therapy via a purposively divergent beam, hence the targeted area and prospective effectiveness of these treatment systems are substantially limited.

There is, therefore, a need for a practical non-invasive acoustic energy delivery system, or treatment imaging probe, which enables an operator to safely and conveniently target therapeutic, high intensity, divergent, high sonic to low ultrasonic frequency acoustic waves at a reasonably high duty factor towards a culprit vascular region through an established skin surface acoustic penetration window. The preferred imaging treatment probe should have a substantially rigid application surface sized and preferably contoured to enable effective seating within a rib space of a patient, particularly for thoracic cavity applications, and should be operable to emit therapeutic high sonic to low ultrasonic acoustic energy with sufficient intensity and duty factor to enable effective penetration and an effective therapy. Ideally, the preferred imaging treatment probe would be configured such that the application surface does not overheat and cause burning of overlying skin and soft tissue of a patient receiving therapy.

### SUMMARY OF THE INVENTION

The present invention provides a non-invasive hand-held treatment imaging probe, which enables the therapeutic delivery of high intensity, high sonic to low ultrasonic frequency acoustic energy to a targeted vascular region, while under ultrasonic imaging guidance via an established acoustic energy penetration window. The treatment imaging probe advantageously comprises a combination of an ultrasonic imaging transducer, and, preferably, a phased array imaging transducer, operatively attached to a therapeutic actuator, the combination configured and sized for hand-held use. In the preferred embodiment, an engagement face of the ultrasonic imaging transducer is operatively disposed about an application end of the therapeutic actuator (operable to emit high energy acoustic energy in about the 1-500 kHz range, and preferably 1-150 kHz range, and most preferably 15-30 kHz range), such that the engagement face and application end of both the ultrasonic imaging transducer and the therapeutic actuator, respectively, share a common application surface on the treatment imaging probe. The application surface of the treatment imaging probe is substantially rigid, and advantageously sized and shaped to enable efficient seating within an intercostal space of a patient, thereby enabling the targeted delivery of high intensity, therapeutic acoustic energy via ultrasonic imaging guidance to the chest wall in thoracic cavity applications. The treatment imaging probe is advantageously designed such that the application surface will not appreciably overheat, and cause burning of the overlying skin or soft tissue of a patient receiving therapy.

The present invention is based on the intuition that in order to ensure adequate penetration and targeting of high sonic to ultrasonic mechanical acoustic transmissions to the heart, guided placement, force and angulation of a transducer with a substantially rigid application surface upon selected chest wall, or rib space, surfaces is required. The chest wall comprises dense intercostal muscle which can severely attenuate acoustic energy propagation, and more importantly, a significant proportion of the coronary anatomy is proximate to or covered by lung, which does not transmit mechanical acoustic energy. For these reasons a directed approach by an operator via imaging guidance, and most conveniently ultrasonic imaging guidance, is a necessity to cardiac applications. A treatment imaging probe sized to enable grasping and manipulation by hand, with an application surface sized and shaped to enable seating within a rib space of a patient, and enabling the combination of both ultrasonic imaging and high intensity, lower frequency therapeutic acoustic energy emissions, enables an operator to apply directed force and angulation to the application surface under imaging guidance to obtain a satisfactory acoustic penetration window and an effective therapy.

The treatment imaging probe is preferably used as an adjunct to clot disruptive and vasodilator therapy in the treatment of acute thrombotic vascular obstructions. It is particularly effective in the treatment of acute ST elevation myocardial infarction as an adjunct to systemically delivered thrombolytic therapy, and/or GP 2b 3a platelet inhibitor therapy, or other dot disruptive and/or vasodilatory therapies to accelerate and ensure thrombolysis.

The application surface of the hand-held and directed treatment imaging probe is applied to the skin surface of a patient, and a viable acoustic energy penetration window and visualization of a vascular target, directly, or by anatomic reference, is established by means of ultrasonic imaging. Once a clear 2-D echo visualization of a target is obtained, such as, for example, the basal aspect of a hypo or akinetic myocardial wall in a heart attack application, where a culprit thrombus is likely to reside, high sonic to low ultrasonic acoustic energy at a high intensity is initiated, such as by a switch or control, through the established and maintained acoustic energy delivery penetration window.

As stated, the preferred therapy is combined with systemically administered clot disruptive and vasodilatory drug therapy, and/or cavitating spheres or microbubbles to accentuate the internal oscillative effect, whereby the therapeutic acoustic effects assist clot disruption, vasodilation, and improved drug interaction and effectiveness to the targeted vascular region. It should be understood that the therapeutic high sonic to low ultrasonic acoustic energy may be imparted continuously, or may be provided in pulses, preferably with a selectable duty factor, in accordance with the desired therapy.

The treatment imaging probe can also be used in similar fashion for the treatment of pulmonary embolus, wherein the treatment imaging probe is directed by ultrasonic imaging to the pulmonary artery, as well as acute peripheral vascular obstructions to the periphery, wherein the culprit blood clot itself, or target vessel may be imaged. The treatment imaging probe could also be used as an alternative treatment for deep vein thrombosis, and for treatment of acute embolic stroke, however in order to direct the therapy past the skull a surgically created acoustic penetration window may be necessary.

Also, in a variation of treatment, such as in a chronic, outpatient setting, the treatment imaging probe may be used to facilitate the localized uptake and delivery of angiogenic agents or other useful medications, which may be, for example, operatively contained within vesicles, wherein the vesicles are designed to rupture and release their contents when exposed to high sonic to low ultrasonic energy.

It is thereby a purpose of the present invention to provide a treatment imaging probe which is practically suitable for therapeutic delivery of high intensity, high sonic to low ultrasonic frequency acoustic energy to a targeted vascular region, while under ultrasonic imaging guidance via an established acoustic energy penetration window.

It is a further purpose of the present invention to provide a treatment imaging probe of the aforementioned type, comprising an ultrasonic imaging transducer operatively attached to a therapeutic actuator operable to emit acoustic energy in about the 1-500 kHz range, and preferably in the 1-150 kHz range, and most preferably in about the 15-30 kHz range, such that the application end of the therapeutic actuator and the engagement face of the ultrasonic imaging transducer share a substantially common application surface, thereby enabling a simultaneous application of high intensity, high sonic to low ultrasonic therapeutic acoustic energy with directed ultrasonic imaging, through an established acoustic penetration window.

It is a further purpose of the present invention to provide a treatment imaging probe of the aforementioned type, which is sized and shaped to enable hand-held manoeuvring and engagement against a contact surface.

It is a further purpose of the present invention to provide a treatment imaging probe of the aforementioned type, wherein the application surface of the instrument is substantially rigid and sized to enable efficient seating within a rib space of a patient, such as to enable optimized imaging techniques and maximized penetration of acoustic energy in thoracic cavity applications.

It is a further purpose of the present invention to provide a treatment imaging probe of the aforementioned type, which is operable to emit a substantially divergent beam of therapeutic acoustic energy at a high duty factor, to ensure targeting and effectiveness of the treatment system wherein the culprit lesion may not always be visible through imaging techniques.

It is a further purpose of the present invention to provide a treatment imaging probe of the aforementioned type, which has means to prevent overheating of the application surface to avoid potential burning of the overlying soft tissue and skin of a patient during therapy.

It is a further purpose of the present invention to define a method of use for the aforementioned treatment imaging probe, to assist in the restoration of blood flow and, optionally assist in drug delivery and drug effectiveness, in a patient experiencing an acute thrombotic vascular obstruction, and in particular an acute thrombotic coronary obstruction.

It is a further purpose of the present invention to define a method of use for the aforementioned treatment imaging probe, for assisted, localized drug delivery of angiogenic drugs or other useful medications, to a compromised vascular system.

### BRIEF DESCRIPTION OF THE DRAWINGS

The apparatus and method of the present invention will now be described with reference to the accompanying drawing figures, in which:

FIG. 1 is a perspective view of a patient lying on his left side receiving treatment from the operator held treatment imaging probe according to the invention;

FIG. 2 is a block diagram of the inter-related components of the treatment system, according to the invention;

FIG. 3 is a perspective view of the treatment imaging probe according to the invention;

FIG. 4 is a simplified diagrammatic presentation of the inner components of the treatment imaging probe according to the invention.

### DETAILED DESCRIPTION

A non-invasive hand-held treatment imaging probe 10 for treating emergency blood flow disturbances and states of low blood perfusion to body regions by imparting therapeutic high sonic to low ultrasonic frequency acoustic energy is described. Treatment imaging probe 10 comprises an ultrasonic imaging transducer 35 operatively attached to a therapeutic actuator 15. The therapeutic actuator 15 is operable in about the 1-500 kHz, and preferably 1-150 kHz, and most preferably 15-30 kHz, frequency range. As stated previously, therapeutic acoustic energy in the high sonic to low ultrasonic frequency ranges is known for its deep penetration characteristics, and superior clot disruptive and thrombolytic enhancement capabilities. Treatment imaging probe 10 has a substantially rigid application surface 12 which is generally sized and shaped to enable seating within a rib space of a patient 20. Application surface 12 advantageously includes an engagement face 32 of ultrasonic imaging transducer 35, and an active end 17 of therapeutic actuator 15, thereby enabling simultaneous therapeutic emissions of acoustic energy in the high sonic to low ultrasonic frequency range while under simultaneous ultrasonic imaging guidance. The preferred application of treatment imaging probe 10 is as an adjunct to thrombolytic and/or other clot disruptive or vasodilatory drug therapy in the emergency room treatment of acute ST elevation myocardial infarction. The administration of thrombolytics, or other useful medications, may be by any known means, however intravenous drug administration is preferred.

High sonic to low ultrasonic frequency acoustic energy provides mechanical agitation via cavitation and acoustic streaming to the blood within the culprit vasculature where a blood clot resides, thereby encouraging disruption of the clot and increased permeation of the drug into clot to accelerate reperfusion. The treatment system is particularly effective for treatment of thrombosis to the coronary, pulmonary, peripheral, and cerebral vasculature.

Treatment imaging probe 10 may also be used on a chronic outpatient basis for the localized delivery and uptake of angiogenic agents (or other useful medications such as anti-restenosis or anti cell proliferation medications), which are of particular use and relevance in cardiovascular systems in patients with known coronary artery disease. In a preferred treatment strategy, the angiogenic agents or other useful medications may be housed within vesicles such as a cavitating spheres or like substances, which are designed to rupture and release their contents when exposed to high sonic to low ultrasonic acoustic energy. The administration of the medications to be delivered, preferably housed within vesicles, may be by any known means, however intravenous drug administration is preferred. In yet another variation of therapy, treatment imaging probe 10 may be used to deliver non invasive sonotherapy to help prevent instent restenosis, with or without the accompaniment of anti restenosis medications.

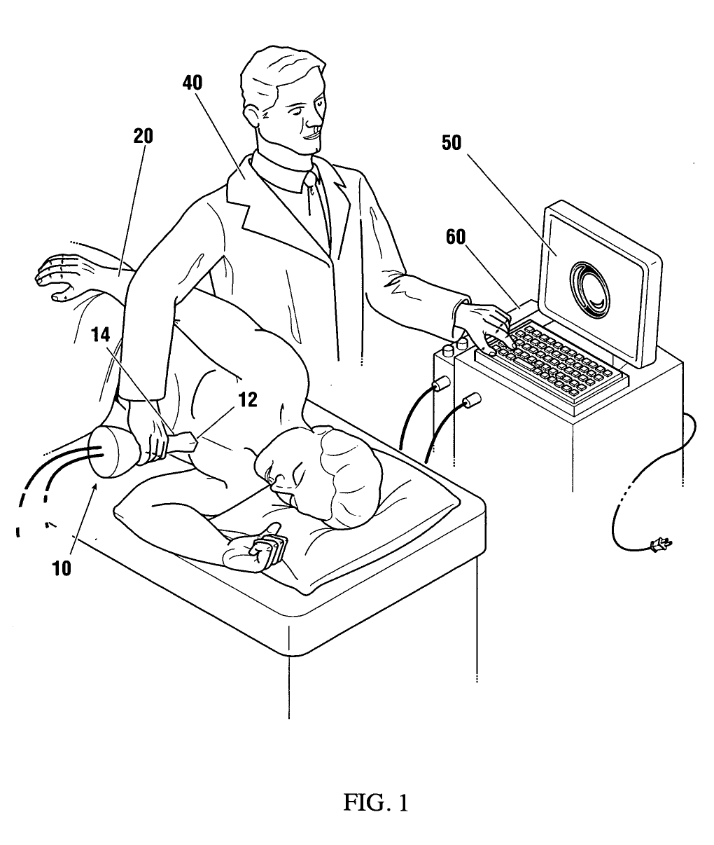

Referring to FIG. 1, a perspective view of the patient 20 receiving treatment from treatment imaging probe 10 according to the invention is shown. IVs and drug administration means are not shown. Treatment imaging probe 10 comprises a housing 14 which is sized to enable grasping by the hand of an operator 40. Referring to FIGS. 1, 2, and 3, the contact surface of treatment imaging probe 10 comprises application surface 12 which shares engagement face 32 of ultrasonic imaging transducer 35 and application end 17 of therapeutic actuator 15. The preferred operational frequency of therapeutic actuator 15 is in the range of about 1 kHz-150 kHz, and most preferably about 15 kHz-30 kHz for maximized penetration and known superior clot disruptive capabilities, and the preferred operational frequency of the ultrasonic imaging transducer 35 is between about 1 MHz-20 MHz for maximal resolution.

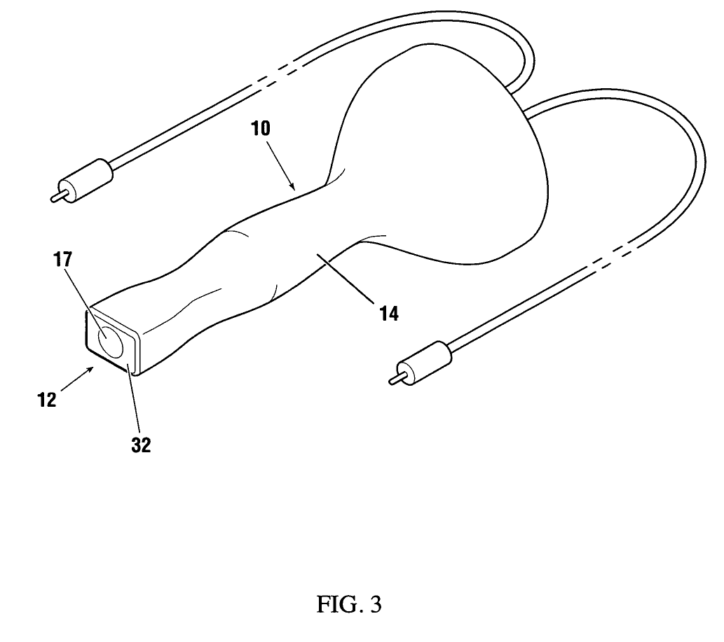

In the preferred embodiment, engagement face 32 of ultrasonic imaging transducer 35 is advantageously disposed circumferentially about application end 17 of therapeutic actuator 15 (see FIG. 3). Other configurations, such as a side by side approach, or where the application end is disposed about the engagement face, may also be used. This latter configuration is beneficial as means may be incorporated to enable focussing of the therapeutic acoustic energy beam, which may be of particular use in applications wherein the culprit blood clot itself can be imaged. Engagement face 32 of ultrasonic imaging transducer 35 may also be partially disposed about application end 17 of therapeutic actuator 15. It is also possible to have the two elements intermixed, or sparsely situated, one within the other wherein smaller piezoelectric crystals, or smaller elements, are used. Ultrasonic imaging transducer 35 advantageously comprises a phased array transducer system, enabling 2D data acquisition and generation of ultrasound beams that can be guided and focussed at many angles and depths, which is ideal for transthoracic cardiac applications. It should be understood that many particular imaging array configurations, phased or otherwise, such as linear, annular, curved, 1D, 1.5D, 2D, sparse 2D, and array configurations enabling 3-D imaging or volume acquisition, may be used depending on the desired therapy, and anticipated nature of the application according to the invention.

The application surface 12 of treatment imaging probe 10 is advantageously sized to enable efficient seating within a rib space of patient 20. This is important to enable effective treatment in applications to the thoracic cavity. In the preferred embodiment, application surface 12 is advantageously of a rectangular shape with a substantially flat or near flat contact surface (see FIG. 3). This shape is especially suited for efficient seating and to supply adequate body surface contact within an intercostal space of patient 20. It should be understood however that the exact shape of application surface 12 is not critical, so long as the resultant application surface is appropriately sized and thereby enables effective seating within a ribspace of an individual in need of therapy. For example, the shape may be circular, oval, square, triangular, trapezoidal, or any other known shape. The contact surface of application surface 12 may be slightly outwardly bowed (or convexly shaped) with respect to housing 14 so as to direct microbubbles away from the patient contact interface, which may otherwise cause unwanted surface heating. Application surface 12 is sized approximately 2.0 cm by 3.4 cm, which is just slightly larger than a typical adult transthoracic imaging probe engagement face and enables superior rib space engagement. While application surface 12 of treatment imaging probe 10 is generally sized to accommodate a vast majority of adult patients, a smaller and a larger variety is optional for extreme cases to suit differing morphologies and rib space sizes.

In use, application surface 12 of treatment imaging probe 10 is applied to the skin surface of patient 20, and a viable acoustic energy penetration window and visualization of a vascular target is established by operator 40 by means of an imaging display of ultrasonic imaging apparatus 50. Once a clear 2-D echo visualization of a target is obtained, such as, for example, the basal aspect of a hypo or akinetic myocardial wall in a heart attack application, wherein the culprit thrombus is likely to reside, and the treatment imaging probe 10 is advantageously pointed towards the vascular target, high sonic to low ultrasonic acoustic energy is initiated, such as by a switch or control, through the pre-established and maintained acoustic energy delivery penetration window. The ideal positioning for patient 20 is on his/her left side in cardiac applications, (FIG. 1), such that the base of the heart drops anatomically leftward and thereby out from under the sternum. However, other positions of the patient 20 may be used, according to the preferred approach. In acute pulmonary emboli applications, such as in saddle emboli, the imaging target comprises the pulmonary artery, and in acute peripheral thrombotic applications the imaging target may comprise the culprit blood clot itself, or at least the target artery. A surgically created acoustic penetration window may be necessary to enable the application of imaging directed therapeutic acoustic energy through the skull in acute embolic stroke applications, where the culprit cerebral vasculature region comprises the imaging target. Treatment of the patient 20 is continued until clinical signs of reperfusion are evident, or alternatively until an invasive correctional procedure, such as emergency angioplasty, is established.

As stated, the preferred therapy is combined with systemically administered clot disruptive and vasodilatory drug therapy, and/or cavitating spheres or microbubbles, whereby the therapeutic acoustic effects assist drug delivery and effectiveness to the targeted region. It should be understood that the therapeutic high sonic to low ultrasonic acoustic energy may be imparted continuously, such as without the need of a control switch, and may have a selectable duty factor, in accordance with the desired therapy. The clot disruptive and vasodilatory drugs may be administered to the patient 20 in any known fashion, however intravenous drug administration is preferred.

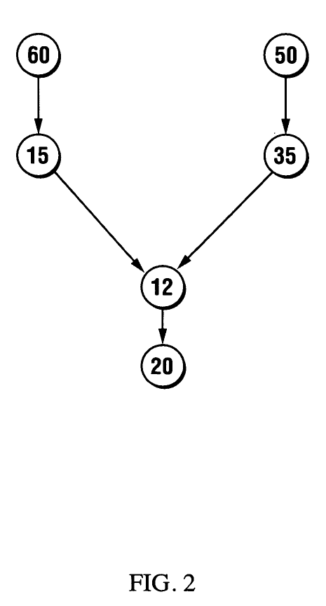

Referring to FIG. 2, a block diagram depicting the various inter-related components of the imaging treatment system in relation to patient 20, is shown. Therapeutic actuator 15 is suitable for human or animal contact and is operatively attached to ultrasonic imaging transducer 35. In the preferred embodiment, engagement face 32 of ultrasonic imaging transducer 35 is circumferentially, or at least peripherally, disposed about application end 17 of therapeutic actuator 15, such that the two elements share a common application surface 12, advantageously sized to enable efficient and preferably contoured seating within a rib space of patient 20 (FIG. 3). Operator 40 controls ultrasonic imaging apparatus 50 and a signal generator 60, such as via an operator interface, which provide electronic signals, and control of ultrasonic imaging transducer 35 and therapeutic actuator 15 respectively.

Referring to FIG. 3, a perspective view of treatment imaging probe 10 is shown. Treatment imaging probe 10 is sized and shaped to enable hand-held engagement and operation. Engagement face 32 of ultrasonic imaging transducer 35, is circumferentially disposed about application end 17 of therapeutic actuator 15, which taken together comprise application surface 12. The peripheral disposition of engagement face 32 about application end 17 enables superior resolution imaging, and once an image of a target is established, an acoustic penetration window, or pathway, for the therapeutic acoustic energy to reach the imaged target is ensured. The piezoelectric elements within engagement face 32 are preferably isolated from the application end 17 of therapeutic actuator 15 by a material having a heavily damped acoustic impedance.

Ultrasonic imaging transducer 35 is made operable simultaneously with therapeutic actuator 15 by providing ultrasonic imaging transducer 35 with a filter for suppressing at least the fundamental acoustic waves emitted from therapeutic actuator 15. The filter advantageously comprises a band rejection, or wedge, filter, wherein the center frequency of the band rejection filter coincides with the emission frequency of therapeutic actuator 15. Therapeutic actuator 15 and ultrasonic imaging transducer 35 can thereby run simultaneously without noteworthy disturbances of the image produced by ultrasonic imaging transducer 35. Alternatively, a high pass filter may be used, wherein the limit frequency thereof essentially coincides with at least, or slightly higher than, the fundamental frequency of the therapeutic acoustic emissions of therapeutic actuator 15. In a variation, such as in the absence of a filter, or in a difficult to image patient, operator 40 may periodically turn off therapeutic acoustic energy, or therapeutic actuator 15, and check the quality of the acoustic penetration window and targeting of the culprit area without interference of the therapeutic high sonic to low ultrasonic frequency acoustic waves.

Application surface 12 is substantially rigid, such as to enable forceful manoeuvring of treatment imaging probe 10 and optional displacement of the skin and soft tissue, and even to a degree the ribs themselves in transthoracic applications, to enable the establishment of the best quality acoustic penetration window possible. In a variation, active end 17 may selectively comprise a semi flexible or flexible membrane, to maximize moulding of active end 17 to the skin surface of patient 20, and thereby maximize penetration of the signal, and minimize the possible collection of air bubbles at the contact interface which can lead to unwanted contact surface heating.

Housing 14 of treatment imaging probe 10 is advantageously cup shaped with an elongated rectangular base, with the cupped portion housing an acoustic generator 19, such as a piezoelectric stack, in the preferred embodiment, and the elongated rectangular base piece housing a cooling chamber 23 filled with acoustic conductive material, which defines a space selectively between the active end of acoustic generator 19 and application end 17, and configured to limit overheating of application end 17, and thereby contact surface 12, during operation. The combination of acoustic generator 19, and cooling chamber 23, and active end 17, substantially defines therapeutic actuator 15. Housing 14 also houses the electronic circuitry and supportive network necessary to operate ultrasonic imaging transducer 35. The elongated rectangular base of housing 14 is advantageously sized and shaped to enable grasping by a hand of operator 40, and is preferably of an hour glass shaped configuration.

Cooling chamber 23 is selectively disposed between acoustic generator 19 and active end 17 of the therapeutic actuator 15, and preferably does not interface with the imaging crystals or operable components of ultrasonic imaging transducer 35, as overlying tissue and skin surface heating effects arise exclusively from the therapeutic actuator 15. It is also preferable to keep the imaging crystals of ultrasonic imaging transducer 35 in direct proximity to the skin surface treated to enable optimal imaging.

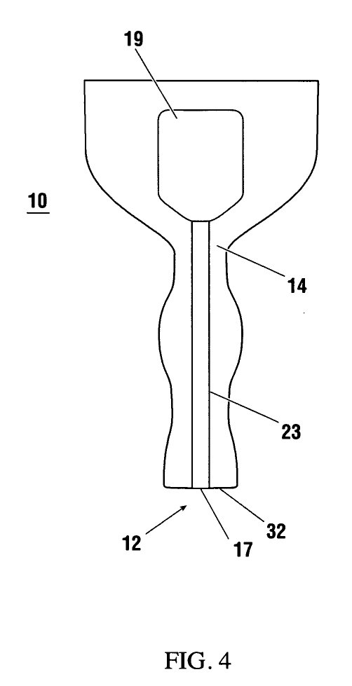

In reference to FIG. 4 a simplified diagrammatic presentation depicting the inner components of treatment imaging probe 10 is shown. As stated previously, housing 14 is generally cupped shaped, with the cupped portion housing acoustic generator 19, and the elongated basal portion housing cooling chamber 23. Cooling chamber 23 is operatively connected to the active end of acoustic generator 19, and adapted to receive and transmit therapeutic acoustic waves to application end 17 of application surface 12. Cooling chamber 23 holds enough acoustic conductive material to enable a substantial heating buffer between the active end of acoustic generator 19 and application end 17, and thereby the skin surface of patient 20. Engagement face 32, which can be used for diagnostic ultrasonic imaging, is shown disposed to either side of active end 17 in this presentation.

The acoustic coupling material housed within cooling chamber 23 is preferably water, and most preferably degassed distilled water, but may comprise any other useful acoustic conducting material such as an ultrasonic gel, oil, or polymer. A cooling sleeve may optionally be disposed exteriorly about housing 14 to limit heating of the acoustic conductive material therein during use. A cooling reactant or reagent may also be provided within housing 14 and/or within cooling chamber 23 intermixed with the acoustic conductive material. The cooling reagent may be in one preferred example, kinetically activated by shaking or agitation.

In a variation, the acoustic coupling material within cooling chamber 23 may be optionally distributed through an inlet passage to cooling chamber 23 and through an outlet passage therefrom, such that the water or other material can circulate through cooling chamber 23. In this variation, herein defined as the “cooling assembly”, the acoustic coupling material may be circulated into cooling chamber 23 from an external tank (not shown), which contains a replenishing supply, or a re-circulated supply, of the acoustic coupling material and may include means for cooling, such as by refrigeration, and/or degassing the medium, such as by vacuum sonification. The acoustic coupling material may alternatively be re-circulated into cooling chamber 23 by an internal tank (not shown), which may for example be housed within the cupped shaped aspect of housing 14. Further, flow management of the acoustic conductive material may include an irrigation manifold in fluid communication with cooling chamber 23, and having means for directing flow of circulating fluid into and out of cooling chamber 23. The irrigation manifold preferably manages the flow of fluid in such a way as to reduce the amount of expelled gasses present in the therapeutic acoustic energy field and additionally provide sufficient dwell time of acoustic conductive material in the field. There are many possible modifications with regards to the construction of an optimal cooling assembly depending on level of detail, cost, and desired result, and an exemplary source of information with regards to various possible constructions and suitable arrangements of cooling mechanisms can be found in U.S. Pat. No. 6,126,619 to Peterson et al., which is incorporated herein by reference, however any other source of information in the art may be used.

Acoustic generator 19 of therapeutic actuator 15 is constructed in accordance to known technology, similar to the acoustic energy applicator disclosed in U.S. Patent Publication No. 2004/0153009 to Horzewski et al., the contents of which are incorporated herein by reference. Cooling chamber 23 of the present invention, which is operatively attached to the active end of acoustic generator 19, to prevent overheating, and enable transmission of therapeutic acoustic waves, is advantageously sized to enable interfacing with active end 17 and seating within an intercostal space of patient 20.

Desirably, an acoustic coupling medium is also applied between application surface 12 and the skin surface of patient 20. The coupling material can comprise a gel material, such as AQUASONIC RTM 100 by Parker Laboratories, Inc., Fairfield, N.J.

Localized skin surface heating effects may arise in the presence of air bubbles trapped between application surface 12 and the skin of patient 20. In the presence of air bubbles the acoustic energy may cause cavitation and result in heating at the skin surface. To minimize the collection of air bubbles along the acoustic contact area, application surface 12 desirably presents an essentially flat or even more preferably outwardly bowed convex radiating surface contour, in other words a surface curved or bowed away from housing 14, where it contacts with or conducts acoustic energy to the skin of patient 20. Given appropriate sizing to enable seating within a rib space, either flat or convex surface contour can mould evenly to the skin topography of patient 20, to thereby mediate against the collection and concentration of air bubbles in the contact area where skin contact occurs. Furthermore, the convex radiation surface advantageously directs air bubbles off the radiating surface. Application surface 12 may also be coated with a hydrophilic material to prevent air bubbles from sticking.

To further mediate against cavitation caused localized skin surface heating, the interior of cooling chamber 23 within housing 14 may be interconnected with at least one, and preferably a plurality of strategically placed recessed well regions (not shown) which are in one embodiment located in higher gravity positions than the active end of acoustic generator 19. Air bubbles that may form in the fluid, or acoustic coupling material, located in cooling chamber 23, are led by gravity to collect in the well region away from the acoustic energy beam path. A slight upward angulation of treatment imaging probe 10, for example, such that application surface 12 is pointed downwards relative to housing 14 during scanning and treatment as shown in FIG. 1, may be considered in this variation. Alternatively, operator 40 may periodically stop treatment and hold treatment imaging probe 10 in a substantially vertical position, such as with application surface 12 pointed downwards, while tapping housing 14 to expedite migration of potential air bubbles to the recessed well regions. The recessed well regions in this example may be controllability partitioned from cooling chamber 23 via a valve, stopcock, or other controllable partitioning mechanism preferably controlled by operator 40, such that the collected bubbles would not escape back into cooling chamber 23 once therapy is resumed. As stated previously, an irrigation manifold may optionally be utilized to expel accumulated bubbles from cooling chamber 23 via an outlet port, or alternatively a reflux valve may be used. The acoustic coupling material is preferably housed within cooling chamber 23 at a relatively high pressure, such as to retard the formation of air bubbles within the acoustic coupling material.

It should be understood that acoustic generator 19 of treatment imaging probe 10 may comprise a variety of piezoelectric generating stacks or a single element may alternatively be used to drive therapeutic actuator 15, and the preferred operational frequency is anywhere in the range of about 1 kHz-150 kHz, and most preferably 15 kHz-30 kHz, according to the invention, although other operating frequencies may be employed, for example, in about the 1 kHz-500 kHz range. The operating intensity of therapeutic actuator 15 is selectable in a range of 0.5 W/sq. cm-25 W/sq. cm, however other intensity selections may be used according to the preferred therapy. Therapeutic acoustic energy with intensity levels of greater than 5 W/sq. cm are generally preferred for transthoracic applications and emergency cases, to ensure adequate penetration. However, if an excellent acoustic penetration window is obtained, lower intensity settings may be considered.

Treatment imaging probe 10 is powered by AC power cord, such as through signal generator 60 and ultrasonic imaging apparatus 50, but may be powered by disposable or rechargeable batteries, such as those in a smaller, more portable imaging based system, to enable use where no AC power source is available.

The ultrasonic imaging transducer 35 is constructed in accordance to known technology, commonly and publicly available to the field of diagnostic ultrasonic imaging. An exemplary supplier of ultrasonic imaging phased array transducers/technology is SIEMENS AG, located in Germany. Similarly, the therapeutic actuator is also constructed according to known technology. An exemplary supplier of therapeutic piezoelectric actuarial systems for human or animal contact is TIMI 3 Systems Inc., located in California USA.

In an example of use, treatment imaging probe 10 is employed as an adjunct to thrombolytic therapy in the treatment of an acute myocardial infarction. Patient 20 presents to the emergency department with severe chest pain and ST elevation is noted on the anterior leads of the 12-lead ECG. An IV is quickly established and TNKase is promptly administered by the attending physician. Operator 40, who may be cardiac ultrasound technician, nurse, or physician trained in delivering diagnostic cardiac ultrasound, is called to administer high sonic to low ultrasonic frequency acoustic wave therapy to accelerate reperfusion and ensure the effectiveness of the thrombolytic drug. Patient 20 is placed on his left side, and operator 40 places treatment imaging probe 10 upon the chest wall surface of patient 20 (FIG. 1). The parastemal views confirm a regional wall motion abnormality in the anterior/anteroseptal wall, which is seen to originate near the level of the base of the heart. The proximal left anterior descending artery (proximal LAD) or Ramus is deemed the likely culprit, and an imaging window depicting the best image of the left ward and lateral aspect of the right ventricular outflow tract and pulmonary artery is obtained, wherein the proximal LAD or Ramus resides by anatomic reference. Continuous wave, high sonic to low ultrasonic acoustic energy is then delivered by operator 40, at a preferred frequency of 27 kHz, and signs of reperfusion are monitored for by the attending nurse or clinician.

In a second example of use, treatment imaging probe 10 is employed in a chronic outpatient setting to improve localized delivery of a systemically administered angiogenic agent to the myocardium and coronary arteries of the heart. Patient 20, who has severe inoperable triple vessel disease and an ejection fraction of about 25%, presents to clinic for localized administration of an angiogenic drug in the hopes of improving his Class 3 angina symptoms. Operator 40, or other care provider, establishes an IV, and administers a therapeutic dose of prepared angiogenic material which has been previously housed within vesicles designed to rupture and release their contents when exposed to high sonic to low ultrasonic mechanical energy. Operator 40, with patient 20 lying on his left side, places treatment imaging probe 10 upon the chest wall surface. An ultrasonic image of the ischemic myocardium is obtained and therapeutic, high sonic to low ultrasonic energy at about 150 kHz is initiated. The vesicles, while circulating systemically about the body, selectively rupture within the vasculature proximate the ischemic myocardium thereby liberating the angiogenic material selectively to the targeted area.

In a variation of angiogenic treatment, such as in cases of triple vessel disease and global ischemia, the imaging/treatment target of treatment imaging probe 10 may comprise the aortic root, such that the angiogenic material, once administered and housed within drug containing vesicles, is liberated within the aortic root and delivered substantially to the coronary circulation. Means for temporarily and selectively increasing the peripheral vascular resistance, such as pressure cuffs, body posturing, or medications, can be employed, to further accentuate coronary flow and selective delivery of the angiogenic material. Optionally, the therapeutic high intensity acoustic waves can be selectively pulsed during the diastolic phase of the cardiac cycle in this variation of treatment, in recognition that the majority of coronary filling occurs during the diastolic phase.

The invention may be embodied in several forms without departing from its spirit or essential characteristics. It should be appreciated that acoustic generator 19 within treatment imaging probe 10 may alternatively comprise a single piezoelectric element, a plurality of smaller piezoelectric elements, a magnetostrictive element, a peristaltic magnetostrictive linear motor, a voice coil, or any other known suitable reciprocating motion generator, according to the invention.

It should also be appreciated that the relative geometry and relative contact surface areas of engagement face 32 of ultrasonic imaging transducer 35 and application end 17 of therapeutic actuator 15 is not critical, as long as both engagement face 32 and application end 17 are represented to a sufficient degree to enable their respective functions, and are placed in close proximity to one another on a substantially rigid, or at least partially rigid, common contact surface. Also, in a variation, application surface 12 may be slightly curved in a convex manner, to better fit the contour of an intercostal space of patient 20 in transthoracic applications, and to better displace potential air bubbles which may form at the skin surface interface.

It should also be appreciated that while the preferred imaging modality of the present invention, i.e. ultrasonic imaging transducer 35, comprises a phased array imaging transducer operatively disposed about active end 17 of therapeutic actuator 15, a variety of types of imaging array configurations, phased or otherwise, such as linear, curved, annular, 1.5D, 2D, sparse 2D or any other type of suitable array or aggregation of imaging crystals, can be used, and placed in any variety of differing relative locations to the therapeutic acoustic wave emission source. For example, a linear array imaging system may be used in conjunction with a therapeutic acoustic wave emission source preferably in between, and most preferably in the middle of, the row of imaging piezoelectric elements provided in the linear array. This arrangement may be particularly suited for imaging and treatment of arteries or veins in peripheral vasculature applications.

It should also be appreciated that more than one treatment head, or application surface 12, may be used, with or without more than one ultrasonic imaging screen, to further optimize penetration to the heart via multiple acoustic penetration windows.

It should also be appreciated that while the preferred treatment imaging probe 10 of the present invention contains a cooling chamber 23 selectively disposed between the active end of acoustic generator 19 and application end 17 of application surface 12, in a variation the active end of acoustic generator 19 may be placed in direct contact with application end 17 without internalized cooling chamber 23, and any optional cooling means for coupling therapeutic acoustic energy to a human body may be disposed exterior to the resultant variant treatment imaging probe. For example, a cooling means may comprise a separate moulded, or non-moulded, exterior component, such as a gel or liquid filled pad or sock, which substantially covers application surface 12, or at least the active end 17, of the variant treatment imaging probe. The exterior component as herein recited may for example comprise its own separate cooling means, such as a fluid exchange mechanism, a cooling agent placed in contact with the exterior component, or possibly a chemical reactant which induces cooling housed within or about the exterior component. These arrangements are satisfactory as long as manoeuvrability of the variant treatment imaging probe and application surface 12 are not unduly compromised and good acoustic penetration windows within the thoracic cavity, such as between the rib spaces, can still be reliably obtained by ultrasonic imaging techniques. In other variations, the cooling means to prevent overheating of application surface 12 can comprise specific actions such as: limiting the intensity of the therapeutic acoustic waves emitted, reducing the duty factor of the therapeutic acoustic waves emitted, alternating or changing acoustic penetration windows, changing instruments, and placing ice or a cold face cloth intermittently over the application area. Preferably, such variations occur under the condition where the active end of acoustic generator 19 is in mechanical contact with the application end 17, or skin surface interface. It should be understood also that any of the aforementioned variations as herein recited may also apply to the preferred treatment imaging probe 10, such as with cooling chamber 23 disposed within housing 14, in the case that extra heating control is deemed necessary.

Finally, treatment imaging probe 10, and all variations thereof, may be operatively attached to a distal active end of a low frequency vibrator operable in the sonic to infrasonic ranges, such as disclosed in co-pending U.S. patent application Ser. No. 10/902,122. This would enable a combination therapy of high amplitude low frequency vibration with high sonic to low ultrasonic acoustic therapy both under ultrasonic imaging guidance, which may be ultimately most preferable in the treatment of acute thrombotic events.

The above-described embodiments of the present invention are intended to be examples only. Alterations, modifications and variations may be effected to the particular embodiments by those of skill in the art without departing from the scope of the invention, which is defined solely by the claims appended hereto.

What is claimed is:

1. A treatment probe comprising,
a) an actuator having a generating end and an application end, said generating end comprising an acoustic generator operable to generate therapeutic mechanical waves,
b) an ultrasonic imaging transducer operatively connected to said actuator wherein the engagement face of said ultrasonic imaging transducer and said application end of said actuator share a substantially common application surface, and
c) a chamber adapted to house an acoustic conductive material, disposed selectively between an active end of said acoustic generator and said application end of said actuator,
whereby said actuator and said ultrasonic imaging transducer enable ultrasonic imaging to direct therapeutic mechanical waves produced by said actuator.

2. The treatment probe of claim 1, wherein said treatment probe is adapted in size and shape to hand-held grasping and manipulation.

3. The treatment probe of claim 1, wherein said application surface is sized and shaped to enable seating in a rib space of a patient.

4. The treatment probe of claim 1, wherein said application surface has a substantially convex shape.

5. The treatment probe of claim 1, wherein said application surface is substantially rigid.

6. The treatment probe of claim 1, wherein said engagement face of said phased array is rigid, and said active end of said actuator is at least partially flexible.

7. The treatment probe of claim 1, wherein said application surface is, at least in part, covered by hydrophilic coating.

8. The treatment probe of claim 1, wherein said acoustic generator is selected from one of: a piezoelectric element, a piezoelectric stack, a magnetostrictive element, a magnetostrictive peristaltic linear motor, and a voice coil.

9. The treatment probe of claim 1, wherein said acoustic generator is operable to generate therapeutic mechanical waves continuously.

10. The treatment probe of claim 1, wherein said acoustic generator is operable to generate therapeutic mechanical waves in pulses.

11. The treatment probe of claim 1, wherein said acoustic generator is operable to emit therapeutic mechanical waves in a substantially divergent beam.

12. The treatment probe of claim 1, wherein said actuator is operable to produce therapeutic acoustic waves at a frequency in the range of about 1-500 kHz.

13. The treatment probe of claim 1, wherein said actuator is operable to produce therapeutic acoustic waves at a frequency in the range of about 1-150 kHz.

14. The treatment probe of claim 1, wherein said actuator is operable to produce therapeutic acoustic waves at a frequency in the range of about 15 kHz-30 kHz.

15. The treatment probe of claim 1, wherein said actuator is operable to produce therapeutic acoustic waves at an intensity level of greater than 5 W/sq. cm.

16. The treatment probe of claim 1, wherein said ultrasonic imaging transducer comprises a phased array imaging transducer.

17. The treatment probe of claim 1, wherein said engagement face of said ultrasonic imaging transducer is operatively disposed about said application end of said actuator.

18. The treatment probe of claim 1, wherein said application end of said actuator is substantially disposed about said engagement face of said ultrasonic imaging transducer.

19. The treatment probe of claim 1, wherein said engagement face of said ultrasonic imaging transducer is disposed side by side with said application end of said actuator.

20. The treatment probe of claim 1, further comprising means for the prevention of overheating of said acoustic conductive material.

21. The treatment probe of claim 1, for use in the treatment of an acute arterial thrombotic obstruction.

22. The treatment probe of claim 1, for use in the treatment of at least one of: an acute coronary syndrome, a pulmonary embolus, an acute peripheral vascular thrombotic obstruction, and an acute cerebral thrombotic obstruction.

23. The treatment probe of claim 1, for use as a means for enhanced clot disruptive or vasodilatory drug delivery in the treatment of an acute thrombotic arterial obstruction.

24. The treatment probe of claim 1, for use in the localized delivery of at least one of an angiogenic agent and an anti-cell proliferation agent to a targeted vascular region.

25. The treatment probe of claim 1, for use in the non-invasive maintenance of stent patency.

26. A method of using the treatment probe as defined in claim 1 for restoring blood flow in a patient experiencing an acute thrombotic vascular obstruction, comprising the steps of:
a) placing the treatment probe as defined in claim 1 to an external body surface proximate said acute thrombotic vascular obstruction,
b) locating an acoustic penetration window enabling visualization of a target of said treatment probe, and
c) applying therapeutic acoustic energy through said acoustic penetration window via said treatment probe to restore blood flow in said patient experiencing said acute thrombotic vascular obstruction.

27. The method of claim 26, wherein said acute thrombotic arterial obstruction is within one of: the coronary vasculature, the pulmonary vasculature, the peripheral vasculature, and the cerebral vasculature.

28. The method of claim 26, wherein said therapeutic acoustic energy is applied at a frequency in the range of about 1 kHz-500 kHz.

29. The method of claim 26, wherein said therapeutic acoustic energy is applied at a frequency in the range of about 1 kHz-150 kHz.

30. The method of claim 26, wherein said therapeutic acoustic energy is applied at a frequency in the range of about 15 kHz-30 kHz.

31. The method of claim 26, wherein said therapeutic acoustic energy has an intensity level greater than 5 W/sq. cm.

32. The method of claim 26, wherein said therapeutic acoustic energy comprises a substantially divergent beam.

33. The method of claim 26, wherein said locating an acoustic penetration window enabling visualization of a target of said treatment probe is accomplished by at least one hand of an operator.

34. The method of claim 26, further comprising the step of administering at least one of: a clot disruptive drug, a vasodilatory drug, and a cavitating microbubble solution; to said patient, prior to completion of step 26 (c).

35. A non-invasive therapeutic probe, comprising,
a) an actuator operable to generate therapeutic mechanical waves in the frequency range of about 1 kHz-500 kHz,
b) an ultrasonic imaging transducer operatively connected to said actuator, wherein an engagement face of said ultrasonic imaging transducer and an application end of said actuator share a substantially common application surface, said application surface sized and shaped to enable efficient seating within a rib space of a patient receiving therapeutic mechanical waves,
whereby said actuator and said ultrasonic imaging transducer enable ultrasonic imaging to direct therapeutic mechanical waves produced by said actuator.

36. The therapeutic probe of claim 35, wherein said actuator is operable in a frequency range of about 1 kHz-150 kHz.

37. The therapeutic probe of claim 35, wherein said actuator is operable in a frequency range of about 15 kHz-30 kHz.

38. The therapeutic probe of claim 35, wherein said actuator is operable to emit therapeutic mechanical acoustic waves at an intensity of greater than 5 W/cm sq.

39. The therapeutic probe of claim 35, wherein said therapeutic probe is adapted in size and shape to enable hand-held engagement and operation.

40. The therapeutic probe of claim 35, wherein said therapeutic probe is operable to emit therapeutic mechanical waves continuously.

41. The therapeutic probe of claim 35, wherein said therapeutic probe is operable to enable therapeutic mechanical waves intermittently, with a selectable duty factor.

42. The therapeutic probe of claim 35, wherein said therapeutic probe is operable to emit therapeutic mechanical waves in a substantially divergent beam.

43. The therapeutic probe of claim 35, wherein said ultrasonic imaging transducer comprises a phased array imaging transducer.

44. The therapeutic probe of claim 35, wherein said application surface is contoured to match said rib space of said patient.

45. The therapeutic probe of claim 35, wherein said application surface is convexly shaped.

46. The therapeutic probe of claim 35, wherein said application surface is, at least in part, rigid.

47. The therapeutic probe of claim 35, wherein said engagement face of said ultrasonic imaging transducer is rigid, and said application end of said actuator is at least partially flexible.

48. The therapeutic probe of claim 35, wherein said application surface is, at least in part, coated by a hydrophilic material.

49. The therapeutic probe of claim 35, wherein said actuator contains an acoustic generator adapted to generate said therapeutic mechanical waves, said acoustic generator comprising at least one of: a piezoelectric element, a piezoelectric stack, a magnetostrictive element, a magnetostrictive peristaltic linear motor, and a voice coil.

50. The therapeutic probe of claim 49, wherein said actuator further comprises a chamber housing an acoustic conductive material, disposed selectively between said acoustic generator and said application end.

51. The therapeutic probe of claim 50, wherein said chamber has an inlet portion and an outlet portion enabling circulation of said acoustic conductive material, such as to enable cooling of said conductive material.

52. The therapeutic probe of claim 35, wherein said engagement face of said ultrasonic imaging transducer is operatively disposed about said application end of said actuator.

53. The therapeutic probe of claim 35, wherein said application end of said actuator is substantially disposed about said engagement face of said ultrasonic imaging transducer.

54. The therapeutic probe of claim 35, wherein said engagement face of said ultrasonic imaging transducer is disposed side by side with said application end of said actuator.

55. The therapeutic probe of claim 35, for use in the treatment of an acute arterial thrombotic obstruction.

56. The therapeutic probe of claim 35, for use in the treatment of at least one of: an acute coronary syndrome, a pulmonary embolus, an acute peripheral vascular thrombotic obstruction, and an acute cerebral thrombotic obstruction.

57. The therapeutic probe of claim 35, for use as a means for enhanced clot disruptive or vasodilatory drug delivery in the treatment of an acute thrombotic arterial obstruction.

58. The therapeutic probe of claim 35, for use in the localized delivery of at least one of an angiogenic agent and an anti restenosis agent to a targeted vascular region.

59. The therapeutic probe of claim 35, for use in the non invasive maintenance of stent patency.

60. An emergency method of using therapeutic acoustic energy for the restoration of blood flow in a patient experiencing an acute coronary obstruction comprising steps of:
a) placing a substantially rigid application surface of a treatment probe upon a chest wall surface proximate the heart, said application surface sized to enable effective seating within a rib space of said patient,
b) locating an acoustic penetration window enabling visualization of a target of therapeutic acoustic energy via use of said treatment probe, and
c) applying therapeutic acoustic energy at a frequency in the range of about 1 kHz-500 kHz through said acoustic penetration window via said treatment probe, to restore blood flow in said patient experiencing an acute coronary obstruction.

61. The method of claim 60, wherein said therapeutic acoustic energy is applied at a frequency in the range of about 1 kHz-150 kHz.

62. The method of claim 60, wherein said therapeutic acoustic energy is applied at a frequency in the range of about 15 kHz-30 kHz.

63. The method of claim 60, wherein said therapeutic acoustic energy has an intensity level greater than 5 W/sq. cm.

64. The method of claim 60, wherein said therapeutic acoustic energy comprises a substantially divergent beam.

65. The method of claim 60, wherein said therapeutic acoustic energy is applied continuously with a duty cycle of 100 percent, during step 60 (c).

66. The method of claim 60, wherein said therapeutic acoustic energy is applied intermittently with a duty cycle of less than 100 percent, during step 60 (c).

67. The method of claim 60, wherein said locating an acoustic penetration window enabling visualization of a target of said treatment probe is accomplished by at least one hand of an operator.

68. The method of claim 60, wherein said target of therapeutic acoustic energy comprises an imaged region within the heart wherein said acute coronary thrombosis is deemed to reside by anatomic reference.

69. The method of claim 60, further comprising the step of administering at least one of: a clot disruptive drug, a vasodilatory drug, and a cavitating microbubble solution; to said patient, prior to completion of step 60 (c).

70. The method of claim 69, wherein said at least one of: a clot disruptive drug, a vasodilatory drug, and a cavitating microbubble solution are administered intravenously.
